(19) United States
(12) Patent Application Publication
(10) Pub. No.: US 2025/0099342 A1
(43) Pub. Date: Mar. 27, 2025

(54) SURFACE MODIFICATION METHOD OF ZIRCONIA MATERIAL USING VACUUM PLASMA

(71) Applicant: AJOU UNIVERSITY INDUSTRY-ACADEMIC COOPERATION FOUNDATION, Suwon (KR)

(72) Inventors: Hee-Kyung Kim, Seoul (KR); ChulHo Kim, Seoul (KR); SungUn Kang, Suwon (KR); Seung-Joo Kim, Suwon (KR); Yu-Kwon Kim, Seoul (KR); Chang-Koo Kim, Seoul (KR); Sanghyun You, Suwon (KR); Dayoung Lee, Pyeongtaek (KR)

(21) Appl. No.: 18/898,326

(22) Filed: Sep. 26, 2024

(30) Foreign Application Priority Data
Sep. 27, 2023 (KR) 10-2023-0131181

(51) Int. Cl. A61K 6/818; A61C 8/00; C04B 35/48; C04B 41/00

(52) U.S. Cl. CPC A61K 6/818; A61C 8/0015; C04B 35/48; C04B 41/0054; C04B 2235/3246; C04B 2235/765

(57) ABSTRACT

The present invention relates to a dental 3Y-TZP zirconia material whose surface is modified by plasma treatment, an implant and a manufacturing method thereof. The 3Y-TZP zirconia material and implant manufactured according to the present invention have improved cell adhesion and antibacterial effects, thereby improving biocompatibility and minimizing inflammatory reactions of the user.

### CROSS-REFERENCE TO RELATED APPLICATION

This application claims priority to and the benefit of Korean Patent Application No. 10-2023-0131181, filed on Sep. 27, 2023, the disclosure of which is incorporated herein by reference in its entirety.

### TECHNICAL FIELD

The present invention provides a surface-modified zirconia material as a method for improving the biocompatibility and antibacterial effect of zirconia. More specifically, the present invention provides a method for modifying the surface of a 3Y-TZP zirconia material implant by treating plasma under vacuum conditions.

The research of the present invention was supported by the Ministry of Science and ICT (grant number: 2019RIF1A1062112 (project unique number: 1711144980) and grant number: 2022R1F1A1067929 (project unique number: 1711172489)), Ministry of Health and Welfare (grant number: HR21C1003 (project unique number: 1465039619)), Ministry of Environment (grant number: ARQ202101282003 (project unique number: 1485019434)) and Ministry of Education (grant number: 2021R1A6A1A10044950 (project unique number: 1345334585)).

### BACKGROUND ART

Dental implants (hereinafter, referred to as “implants”) are artificial teeth that can permanently replace missing teeth, and thus, they must be able to functionally perform the role of real teeth. In addition, they must be manufactured such that the load applied to the teeth during chewing can be appropriately distributed, and thus, they can be used for a long period of time, and they must be delicately manufactured such that they have a shape and color that are not much different from real teeth in terms of aesthetics.

Implants are implanted and fixed into the oral tissue, that is, the alveolar bone, and after being implanted into the body, metal ions from the metal implant are dissolved by tissue fluid or body fluid in the body or by contact and friction with the body tissue, thereby causing the implant to corrode. In addition, metal ions dissolved from the metal implant can damage macrophages in the body or invade cells in the body, thereby causing the generation of inflammatory cells or giant cells, and thus, the implant must have excellent biocompatibility.

Since implants are required to satisfy conditions related to biocompatibility and chemical compatibility, such as osseointegration, research on implants is mainly being conducted on fixtures corresponding to the roots. In particular, since the fixture part is mainly made of titanium or some titanium alloys with excellent biocompatibility, research on promoting the bonding between titanium materials and bone or dispersing stress, such as surface treatment technology using titania (TiO₂) nanotubes, is being actively conducted.

However, since procedures using such implants have the possibility of bacterial infection due to surgical operations, the bone around the implant may melt due to the bacterial infection, thereby causing the implant to be lost, or side effects such as pain, inflammation or swelling may occur, and these side effects are causing greater problems in implant treatment. Accordingly, research on methods that can enhance the antibacterial effect of implants while improving cell adhesion is necessary.

### DISCLOSURE

### Technical Problem

The present invention is directed to providing a dental 3Y-TZP zirconia material or a dental 3Y-TZP zirconia implant having improved chemical activity, cell adhesion and antibacterial effect by surface modification.

The present invention has discovered plasma conditions that are effective for improving the cell adhesion and antibacterial effect of a 3Y-TZP zirconia material and a dental zirconia implant and treated the same with 3Y-TZP zirconia, and is directed to providing an implant made of a 3Y-TZP zirconia material having improved stability, antibacterial effect, cell proliferation, cell mobility and cell adhesion.

In addition, the present invention is directed to providing a plasma treatment process for manufacturing a surface-modified 3Y-TZP zirconia material and a dental zirconia implant.

However, the technical problems to be solved by the present invention are not limited to the problems mentioned above, and other problems that are not mentioned will be clearly understood by those skilled in the art from the description below.

### Technical Solution

Hereinafter, the present invention will be described in detail. The advantages and features of the present invention and the exemplary embodiments that achieve the same will be clearer with reference to the exemplary embodiments described below. However, the present invention is not limited to the exemplary embodiments disclosed below, but can be implemented in various different forms, and these exemplary embodiments are provided only to make the disclosure of the present invention complete and to fully inform those skilled in the art to which the present invention pertains of the scope of the invention, and the present invention is defined only by the scope of the claims. Like reference numerals refer to like elements throughout the specification.

Unless otherwise defined, all terms (including technical and scientific terms) used in the present specification may be used in meanings that can be commonly understood by those skilled in the art to which the present invention pertains. In addition, terms defined in commonly used dictionaries are not to be ideally or excessively interpreted unless explicitly specifically defined. The terms used in the present specification are for the purpose of describing the exemplary embodiments and are not intended to limit the present invention. In the present specification, singular forms also include plural forms unless specifically stated in the phrase.

The present invention provides a 3Y-TZP zirconia material whose surface is modified by plasma treatment, a dental zirconia implant made of the zirconia material, and a dental zirconia implant whose surface is modified by plasma treatment.

The zirconia material and dental zirconia implant whose surface is modified according to the present invention have improved cell proliferation, adhesion and motility, and have improved antibacterial effects. In addition, plastic deformation occurs on the surface such that chemical activity and reactivity are high, and the C/O ratio on the surface is low, and thus, reactivity with cells is high.

In addition, the present invention provides a method for modifying the surface of a 3Y-TZP zirconia material, including the steps of (a) filling a carrier gas into a plasma generating device; (b) generating plasma in the plasma generating device; and (c) irradiating the generated plasma onto zirconia.

According to another exemplary embodiment of the present invention, provided is a method for manufacturing a dental implant whose surface is modified, including the steps of (a) preparing an implant composed of a 3Y-TZP zirconia material; (b) filling a plasma generating device with a carrier gas; (c) generating plasma in the plasma generating device; and (d) irradiating the generated plasma to the implant of step (a).

In the present invention, the zirconia material or implant is preferably for medical use, and more specifically, for dental use.

The zirconia material or implant whose surface is modified according to the present invention may have a surface C/O ratio of 1 to 3.

In addition, the plasma of the present invention may be formed or radiated under vacuum conditions, and preferably, the plasma may include N₂ or N₂/Ar as a carrier gas (consisting of N₂ or N₂/Ar gas). In this case, the mixed gas of N₂/Ar preferably includes N₂ and Ar at a molar ratio of 1 to 5:5 to 50.

The material or implant whose surface is modified according to the present invention may be treated with plasma for 0.1 seconds to 10 minutes (preferably, 0.1 seconds to 5 minutes).

The plasma of the present invention may be generated by supplying a voltage of 0.1 kV to 40 kV (preferably, 0.1 kV to 20 kV) and a frequency of 10 to 30 kHz to the plasma generating device, and may be radiated under room temperature and vacuum conditions. The plasma of the present invention is preferably irradiated to the zirconia for 0.1 seconds to 10 minutes.

### Advantageous Effects

According to the present invention, the surface-modified 3Y-TZP zirconia material and zirconia implant have improved cell proliferation, adhesion and motility, and have improved antibacterial effects.

In addition, the 3Y-TZP zirconia material and the implant of the 3Y-TZP zirconia material according to the present invention have reduced contact angles of water and diiodomethane, and plastic deformation occurs on the surface, resulting in high chemical activity and reactivity. In addition, the 3Y-TZP zirconia material of the present invention has a low C/O ratio on the surface, and thus has high reactivity with cells, thereby making it suitable for use as a dental material.

The present invention provides the most effective plasma type and generation method for modifying the surface of the 3Y-TZP zirconia material.

According to the method for manufacturing a surface-modified 3Y-TZP zirconia material and implant according to the present invention, the antibacterial activity and cell adhesion of the implant are improved, and thus, there is an advantage that it is possible to minimize inflammatory reactions resulting from the use of the implant.

In addition, the implant according to the present invention forms a hydrophilic moisture film, thereby improving the success rate of implant surgery and shortening the surgery period.

The surface-modified zirconia material according to the present invention or the implant using the same can be used for purposes such as antibacterial (sterilization) use, cell proliferation use and cell attachment use.

### DESCRIPTION OF DRAWINGS

FIG. 1 shows changes in the water contact angle according to the plasma treatment time of atmospheric pressure plasma and vacuum plasma for a zirconia material according to an exemplary embodiment of the present invention.

FIG. 2 shows the XPS spectrum analysis results according to an exemplary embodiment of the present invention.

FIG. 3 shows the surface texture parameters (Sa, arithmetic mean height; Sq, root mean square height; Sv, maximum pit height) of all experimental groups obtained by using a confocal laser scanning microscope (CLSM) according to an exemplary embodiment of the present invention.

FIG. 4 shows the SEM image (left) at 40,000× magnification and the Fib cross-section image (right) of the cross-section at 6,000× magnification of each experimental group according to the present invention.

A of FIG. 5 shows the X-ray diffraction patterns of all experimental groups according to an exemplary embodiment of the present invention, and B and C of FIG. 5 show the Rietveld quantitative analysis results as a function of plasma exposure time for the vacuum plasma treatment group (B) and the atmospheric pressure plasma treatment group (C). D of FIG. 5 shows the cubic unit cell parameter (Å) as a function of plasma exposure time.

FIG. 6 shows changes in the zeta potential according to plasma treatment time by using atmospheric pressure plasma and vacuum plasma according to an exemplary embodiment of the present invention.

FIG. 7 is a schematic diagram showing an experimental process for treating a zirconia specimen with plasma according to an exemplary embodiment of the present invention.

### MODES OF THE INVENTION

Hereinafter, in order to help understanding of the present invention, examples will be provided to describe in detail. However, the following examples are only intended to illustrate the content of the present invention, and the scope of the present invention is not limited to the following examples. The examples of the present invention are provided to more completely explain the present invention to a person having ordinary skill in the art.

The present invention is directed to providing a zirconia material having improved cell proliferation ability, adhesion ability and motility, and having improved antibacterial effect. The zirconia material of the present invention has reduced contact angles of water and diiodomethane according to plasma treatment, and plastic deformation occurs on the surface, and thus, it has high chemical activity and reactivity. In addition, the zirconia material of the present invention has a low C/O ratio on the surface, and thus, it has high reactivity with cells.

The “zirconia” of the present invention is not limited to its type, but is preferably 3Y-TZP zirconia.

In the present invention, “3Y-TZP (3 mol % yttria-stabilized tetragonal zirconia polycrystal)” means a 3 mol % yttria-stabilized tetragonal zirconia polycrystal with high mechanical strength.

In the present invention, “plasma” can be generated by supplying a specific pressure, voltage or frequency to a carrier gas. The “carrier gas” in the present invention may be at least one gas selected from the group consisting of nitrogen, helium, argon and oxygen, but the present invention is not limited thereto. Preferably, it may be composed of a mixture of at least two types of gases among nitrogen, helium, argon and oxygen, but the present invention is not limited thereto, and most preferably, it may be nitrogen (N₂) or a mixture of nitrogen and argon (N₂/Ar). In the present invention, the mixture of nitrogen and argon may be a mixture of N₂ and Ar at a molar ratio of 1 to 5:5 to 50, and preferably, a mixture at a molar ratio of 1:9.

The plasma of the present invention may be generated by applying a voltage of 0.1 kV to 20 kV and/or a frequency of 10 to 30 kHz to the carrier gas. Most preferably, it may be generated by applying a voltage of 5 kV and/or a frequency of 25 kHz.

In the present invention, “surface modification” means imparting physical, chemical and biological properties that were not present in the original material to the surface of a material, and in the present invention, it means that the contact angle of water and the contact angle of diiodomethane on the surface of a zirconia material decrease by plasma treatment, plastic deformation occurs on the surface, chemical activity and reactivity increase, and the C/O ratio decreases, thereby increasing reactivity with cells.

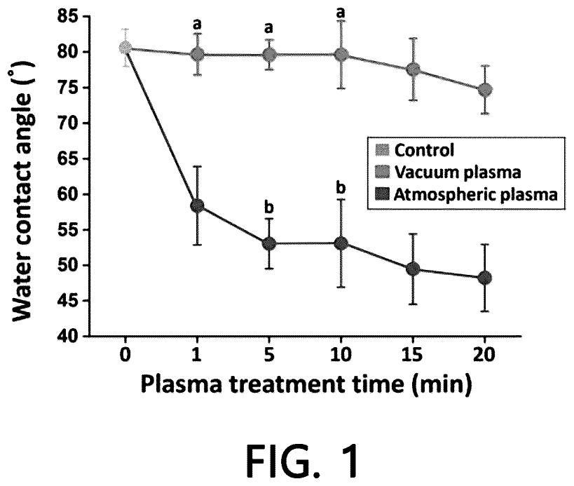

According to an exemplary embodiment of the present invention, the contact angle of water and the contact angle of diiodomethane on the surface of the zirconia material decrease overall by plasma treatment (refer to FIG. 1). In particular, the surface free energy increases during plasma treatment, and among these, the polar component (γ^{P}) increases in the plasma (as a carrier gas) consisting of N₂/Ar (refer to Table 1), and thus, it can be seen that the zirconia material whose surface is modified according to the present invention has improved cell adhesion.

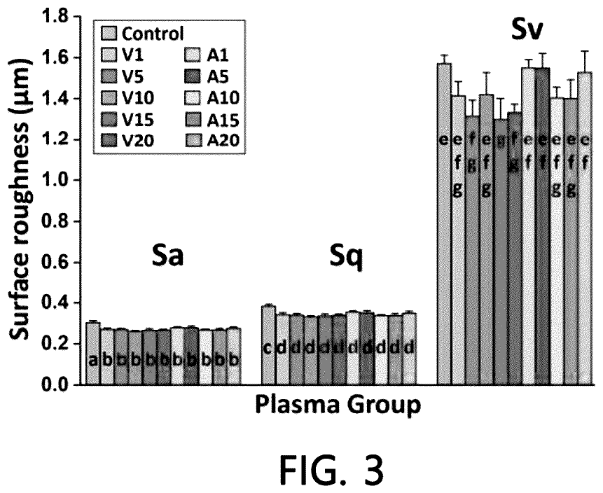

In addition, according to an exemplary embodiment of the present invention, in the case of zirconia whose surface is modified by plasma treatment, there was no significant difference in surface roughness (refer to FIG. 3), but plastic deformation occurred in the plasma (as a carrier gas) consisting of N₂/Ar, and thus, oxygen uptake is accelerated on the surface of the modified material, and chemical reactivity and kinetics of surface reactions are increased.

The surface of zirconia whose surface is modified according to the present invention may have a different composition depending on the plasma treatment. According to an exemplary embodiment of the present invention, when treated with plasma using N₂/Ar as a carrier gas, a change in the percentage of O atoms between lattice oxygen (OL), acidic hydroxyl OH(a) and basic hydroxyl OH(b) on the surface of the zirconia occurs, and an increase in the percentage of oxygen atoms and an increase in the percentage of nitrogen atoms occur.

In the case of the zirconia material whose surface is modified according to the present invention, the antibacterial effect increases, and the bacterial adhesion ability decreases, thereby making it suitable for use as a dental material. The zirconia material of the present invention has an antibacterial effect on bacteria existing in the oral cavity, and has an antibacterial (sterilizing) effect on at least one bacteria selected from the group consisting of Porphyromonas gingivalis, Tannerella forsythia and Treponema denticola.

In addition, the zirconia material prepared according to the present invention is suitable for use as a medical material, because it has improved cell proliferation, cell motility and cell adhesion in its relationship with cells (preferably, osteoblasts). As described above, the zirconia material of the present invention has improved cell proliferation, motility and adhesion in its reaction with osteoblasts, and thus, when used as a dental material, the efficacy of bone formation and osseointegration is improved.

As used herein, the term “treatment” of plasma means irradiating plasma to zirconia (or a specific material made of zirconia). In the present invention, the plasma may be radiated at room temperature and under vacuum conditions, and may be irradiated to the zirconia for 0.1 seconds to 10 minutes, and preferably, for 60 seconds (more preferably for 0.1 seconds to 5 minutes) at a distance of 10 mm.

The “zirconia material” of the present invention may be used as a dental material, and while the present invention is not limited thereto, it may be used for implants, crowns, inlays, posts and orthodontic brackets, and most preferably, it may be used as a dental implant material.

In the present specification, the term “implant” means a replacement that restores lost human tissue, and a dental implant generally means a replacement that is implanted into the alveolar bone where the natural tooth root has fallen out to replace the root of a lost tooth, and then, an artificial tooth is fixed on top thereof to restore the original function of the tooth.

In the present invention, the “dental zirconia implant whose surface is modified by plasma treatment” may be made of the zirconia material whose surface is modified, or may be manufactured by treating an implant made of zirconia with plasma.

Hereinafter, the present invention will be described in more detail through examples. These examples are intended only to illustrate the present invention, and it is apparent to those skilled in the art that the scope of the present invention is not to be construed as being limited by these examples.

### EXAMPLE 1

### 1-1. Sample Preparation and Plasma Surface Treatment

A total of 198 sintered 3Y-TZP specimens (KATANA ML, Kuraray Noritake Dental, Osaka, Japan) measuring 10.0 mm×10.0 mm×1.0 mm were used in this example. All specimens were polished by using 600 to 1,200 grit SiC abrasives and then cleaned in an ethanol ultrasonic bath for 5 minutes. The 3Y-TZP specimens were randomly divided into two main groups: vacuum plasma (V) and atmospheric plasma (A) according to the chamber gas pressure. Each group was subdivided into five subgroups according to the plasma treatment time (1, 5, 10, 15 and 20 minutes): V1, V5, V10, V15, and V20 for vacuum plasma; and A1, A5, A10, A15 and A20 for atmospheric plasma. Specimens of each experimental group were exposed to a N₂/Ar gas mixture (10% N2 and 90% Ar) for various treatment times under atmospheric pressure or vacuum. The control group was not subjected to plasma treatment.

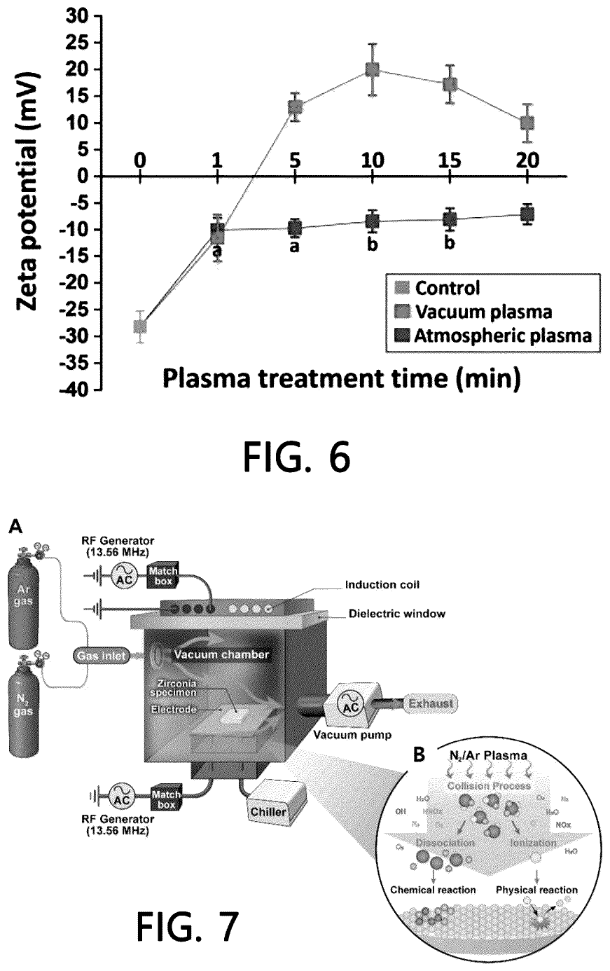

For the low-pressure group, a planar ICP source (ICP system, Samvac Co., Paju, Republic of Korea) was supplied with 150 W, −200 V bias AC at a 13.56 MHz radio frequency under a 4 Pa vacuum pressure. A low-frequency (30 Hz) DBD system (PR-ATO-001, ICD Co., Anseong, Republic of Korea) using AC voltage was used to generate air plasma. The distance between the plasma nozzle tip and the specimen surface was maintained at 10 mm. The experimental conditions and schematic diagram of the plasma treatment for the zirconia specimens in this example are as shown in FIG. 7.

(A) of FIG. 7 shows the ICP vacuum plasma system, and (B) of FIG. 7 shows the surface modification mechanism according to the plasma treatment of 3Y-TZP.

### EXAMPLE 2

Analysis of the Physical Properties of the Zirconia Surface after Plasma Treatment

### 2-1. Confirmation of Changes in Surface Contact Angle and Surface Free Energy According to Plasma Treatment

The surface free energy (γ) was obtained by measuring the contact angle of three test liquids (water, glycerol and diiodomethane) deposited on the zirconia surface by using a contact angle analyzer (Phoenix 300 Touch, S.E.O., Suwon, Republic of Korea).

According to the Lifshitz-van der Waals (LW) acid-base method, γ can be divided into the additive Lifshitz-van der Waals (LW) component and the Lewis acid-base (AB) component, as shown in Formula 1 below:

γ=γ^{LW}+γ^{AB}=^{LW}+2\sqrt{γ⁺γ⁻} <Formula 1>

In Formula 1 above, γ^{LW} includes all the electrodynamic dispersion forces, and the acid-base component (γ^{AB}) is decomposed into electron donor (Y) and electron acceptor (Y+) parameters.

### 2-2. X-Ray Photoelectron Spectroscopy

X-ray photoelectron spectroscopy (XPS) analysis was performed to compare the surface chemical changes of zirconia treated with air and vacuum plasma at various treatment times. The measurements were performed on the core levels of C 1s, O 1s, N 1s, Y 3d and Zr 3d regions by using XPS (K-alpha, Thermo Scientific Inc., UK) equipped with a monochromatic Al Kα X-ray source (1486.6 eV) at 12 kV. The spectra were aligned to the C Is peak at 284.6 eV as a reference. The compositional depth profile of the plasma-treated zirconia surface was measured by using Ar+ ion sputtering excited at 2 keV energy with a sputtering rate of 0.30 nm/s and a total sputtering time of 60 s, and the penetration of nitrogen ions after plasma treatment was determined.

### 2-3. Changes in Surface Topography

The changes in the three-dimensional surface topography after plasma irradiation were analyzed by using CLSM (LEXT OLS3000, Olympus, Tokyo, Japan) with a measurement area of 256×192 μm². The surface texture parameters (Sa, Sq and Sv) were determined according to the ISO 25178 reference. Ten measurement values were obtained for each group.

### 2-4. SEM and FIB Analysis

The microstructure of the zirconia surface after plasma treatment was characterized by SEM (JSM-7800F Prime, JEOL, Tokyo, Japan) at magnifications of 2000×, 10,000× and 40,000×. EDS spectra were obtained together with SEM by using an EDS detector (X-max 150, Oxford Instruments NanoAnalytics, High Wycombe, UK) to identify the local chemical composition. Dual-beam cross-sectional analysis was performed by using FIB/SEM imaging to investigate the subsurface structure. Milling was performed at a current of 300 pA by using gallium ions accelerated at 30 kV.

### 2-5. X-Ray Diffraction (XRD) and Rietveld Analysis

Quantitative identification of the crystal phases in each experimental group was determined by the Rietveld refinement method by using XRD (DMAX-2200PC, Rigaku, Tokyo, Japan) and CuKα radiation at 40 kV and 30 mA. XRD profiles were obtained at room temperature in the 20 range of 10 to 100° with a step size of 0.02° and a calculation time of 2 seconds per step. Structure refinement was performed by the Rietveld method using the Fullprof program. The diffraction profiles were fitted with the Pseudo-Voigt peak function and manually selected background points.

### 2-6. Zeta Potential Measurement

Zeta potential analysis was evaluated by the electrophoretic light scattering technique in 10 mM NaCl (pH 5.6) using an electrokinetic analyzer (Mastersizer 3000, Malvern Panalytical Ltd., Malvern, UK), and was measured five times at 25° C.

### 2-7. Statistical Analysis

Statistical analysis of the data was performed by using two-way ANOVA to determine the effects of two independent variables, that is, chamber pressure and plasma treatment time, on the contact angle, surface roughness and zeta potential of 3Y-TZP. The analysis was performed by using a software suite (IBM SPSS Statistics, v25.0, IBM Corp., Chicago, IL, USA), and a p-value less than 0.05 was considered statistically significant.

### EXAMPLE 3

### Results

### 3-1. Analysis of Surface Free Energy (SFE) Components

A two-way analysis of variance (ANOVA) revealed a statistically significant interaction between chamber pressure and plasma treatment time with respect to surface contact angle (p<0.05). The changes in water contact angle according to plasma treatment time are shown in FIG. 1.

FIG. 1 shows changes in the water contact angle according to plasma treatment time of atmospheric pressure plasma and vacuum plasma according to this example. In both plasma treatment groups, the contact angle decreased as the treatment time increased. The means within each plasma system indicated by the same letter in FIG. 1 are not significantly different from each other (p>0.05).

In both plasma treatment groups, the contact angle decreased as the treatment time increased, but the atmospheric pressure plasma treatment group showed a lower value in the range of 44.8 to 64.7° than the vacuum plasma treatment group. The surface energy components of all experimental groups based on the probe liquid are as shown in Table 1 below. When calculated by using the three-liquid method, the SFE of 3Y-TZP increased with plasma treatment in all groups, which contributed to an increase in the polar component (γAB). The total surface energy is a result of electrodynamic interactions dominated by acid-base interactions rather than dispersion forces. As the plasma treatment time increased, the SFE showed a tendency to slightly increase. In particular, the highest SFE value was obtained in V15. Plasma treatment of the zirconia surface increased the electron donating (γ⁻) capacity under atmospheric pressure, whereas plasma treatment decreased the γ⁻ parameter in the vacuum plasma-treated group as the treatment time increased.

TABLE 1

| | | | | | |
|---|---|---|---|---|---|
| Material | γ | γ^{LW} | γ^{AB} | γ⁺ | γ⁻ |

Liquid

| | | | | | |
|---|---|---|---|---|---|
| DI water^{a} | 72.80 | 21.80 | 51.00 | 25.50 | 25.50 |
| Glycerolª | a 63.40 | 34.00 | 29.40 | 3.92 | 57.00 |
| Diiodomethane^{a} | 50.80 | 50.80 | 0.00 | 0.00 | 0.00 |

Group

| | | | | | |
|---|---|---|---|---|---|
| Control | 17.51 | 41.40 | −23.89 | 5.98 | 23.84 |
| V1 | 33.98 | 39.77 | −5.79 | 0.28 | 29.99 |
| V5 | 34.62 | 35.56 | −0.94 | 0.02 | 9.91 |
| V10 | 34.63 | 36.66 | −2.03 | 0.11 | 9.71 |
| V15 | 39.17 | 37.44 | 1.74 | 0.10 | 7.83 |
| V20 | 30.64 | 35.61 | −4.97 | 0.35 | 17.56 |
| A1 | 30.74 | 41.36 | −10.61 | 0.69 | 40.82 |
| A5 | 34.97 | 39.65 | −4.67 | 0.14 | 38.37 |
| A10 | 36.62 | 37.48 | −0.86 | 0.01 | 33.02 |
| A15 | 36.57 | 39.65 | −3.08 | 0.06 | 42.17 |
| A20 | 31.94 | 40.53 | −8.59 | 0.40 | 45.68 |

In Table 1, the surface energy components of all experimental groups are based on the probe liquid, and the unit of the shown values is mJ/m². The superscripts LW and AB describe Lifshitz-van der Waals and Lewis acid-base interactions (electron acceptor, γ⁺/donor, γ), respectively. The ‘a’ value refers to van Oss (Van Oss, C. J. Long-range and short-range mechanisms of hydrophobic attraction and hydrophilic repulsion in specific and aspecific interactions. J. Mol. Recognit. 2003, 16, 177-190.).

### 3-2. Surface Chemical Analysis

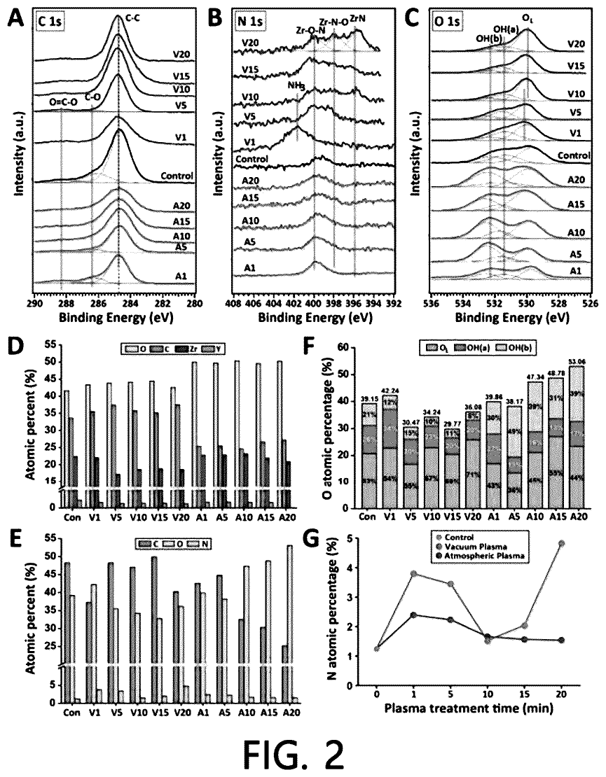

In FIG. 2, the XPS spectra show the C1s (FIG. 2A), N 1s (FIG. 2B) and O 1s (FIG. 2C) regions of all experimental groups according to the exemplary embodiment of the present invention, and D of FIG. 2 shows the chemical composition obtained from the SEM-EDS analysis. E of FIG. 2 shows the atomic percentages of C, N and O on the zirconia surface, which were obtained from the XPS spectra. F of FIG. 2 shows the relative ratios of lattice oxygen (OL), OH(a) and OH(b) in the O1s core level XPS spectrum, and G of FIG. 2 shows the N atomic percentage as a function of plasma treatment time, which was obtained from the XPS analysis.

Specifically, A of FIG. 2 shows the X-ray photoelectron spectroscopy (XPS) C1s spectra of all experimental groups. The spectrum of the control specimen can be decomposed into three components: a component at 284.8 eV due to the C—C bond of the adventitious carbon layer, a component at 286.4 eV due to the C—O bond, and a component at 288.4 eV due to the O═C—O bond. As the plasma treatment time increased, the intensity of the C—C peak decreased, whereas the intensities of C—O and O═C—O increased, indicating that the zirconia surface was oxidized by reactive oxygen species (ROS) after atmospheric pressure plasma irradiation. The plasma ions broke the C—C bonds to form C radicals, which combined with ROS such as O⁺, O₂⁺, O₂⁺ and O₂²⁺. As illustrated in C, E, and F of FIG. 2, the atmospheric pressure plasma induced the generation of a higher concentration of oxygen functional groups that could break the C—C bonds compared to the vacuum plasma.

The N1s photoelectron region (FIG. 2B) showed a characteristic component at a binding energy of 400 eV, which represents the N of zirconium oxynitride or ZrOₓN_{y}. This means that zirconium oxynitride was formed in the near-surface region of all plasma groups. The V20 group shows the bonding configuration of N in zirconium nitride (ZrN) at a binding energy of 396 eV, indicating the formation of the ZrN layer. There is a possibility that the N concentration in V20 may have reached the critical concentration required for the formation of the ZrN layer.

The XPS spectrum of O1s is shown in C of FIG. 2. The peak at approximately 530 eV belongs to the lattice oxygen (OL) of ZrO₂, and the peaks at approximately 531.5 and approximately 532.5 eV are attributed to the O components associated with the acidic hydroxyl group OH(a) and the basic hydroxyl group OH(b), respectively. F of FIG. 2 shows the quantitative ratios of lattice oxygen (OL), OH(a) and OH(b) in the O Is core level spectra for all experimental groups. The atmospheric pressure plasma promoted the formation of the OH(b) group, which is the surface oxygen chemically adsorbed on the zirconia surface (FIG. 2F). The binding energy of OL shifted to lower energy by using vacuum plasma (C in FIG. 2), which means that lattice oxygen was more ionized in nature.

The chemical composition (approximately 1 μm deep) obtained by energy dispersive X-ray spectroscopy (EDS) analysis showed that O, C, Zr and Y elements were present on the surface (D in FIG. 2). According to the scanning electron microscope (SEM)-EDS results, the amount of O element increased in the atmospheric pressure plasma treatment group, whereas the amount of C element decreased. SEM-EDS analysis limits nitrogen detection due to the low efficiency of low Z elements. XPS results (E, F in FIG. 2) showed that the percentage of O element content increased with increasing treatment time in the atmospheric pressure plasma treatment group. XPS, which is a surface-sensitive technique with an estimated penetration depth of several nanometers, indicates that the N element was injected into the outermost surface layer after plasma treatment. Changes in the N atomic percentage with plasma treatment time are shown in G in FIG. 2. The N concentration increased rapidly after 1 minute and then decreased in both plasma treatment groups. In the vacuum plasma treatment group, the N atomic percentage decreased to a value of 1.51% and then increased. Depending on the plasma type and plasma exposure time, the N species were integrated to approximately 17 nm, and the vacuum plasma treatment and increased treatment time tended to improve nitrogen diffusion.

### 3-3. Surface Characteristics

FIG. 3 shows the surface texture parameters (Sa, Sq and Sv) of all experimental groups. No significant differences in Sa, Sq and Sv were observed in all plasma treatment groups, and the means of groups indicated by the same letters in FIG. 3 were not significantly different from each other (p>0.05).

FIG. 3 shows the surface texture parameters (Sa, arithmetic mean height; Sq, root mean square height; Sv, maximum pit height) of all experimental groups obtained by using a confocal laser scanning microscope (CLSM). All plasma treatment groups showed a slight decrease in value, which can be considered a mild etching effect that smoothes the surface, although it was very low. In addition, although the roughness values decreased slightly in the vacuum plasma treatment group, no statistically significant differences were observed in the Sa and Sq values among the plasma treatment groups.

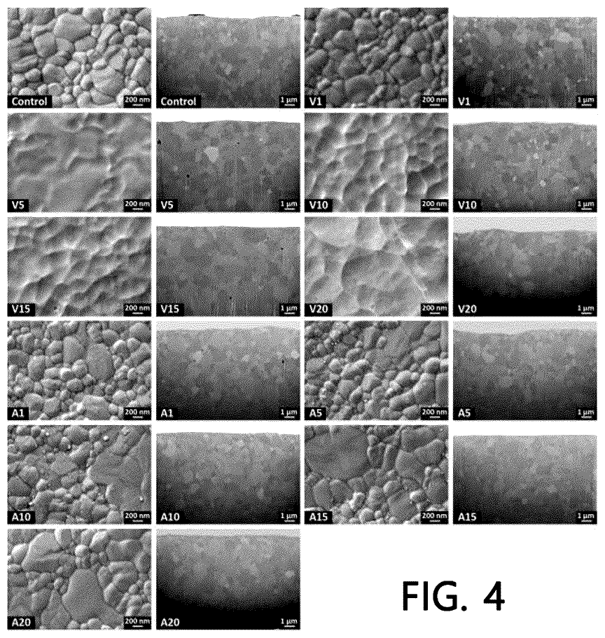

FIG. 4 shows the SEM image at 40,000× magnification (left) and the Fib cross-section image at 6,000× magnification (right) of each experimental group. The vacuum plasma caused surface erosion due to electric discharge, but the microstructure beneath the surface was not changed.

The SEM micrograph (FIG. 4) shows that the grain boundaries were degraded under high electric fields in the vacuum plasma treatment group. The SEM images also show surface erosion due to energetic ion bombardment of the high-power pulsed plasma stream. However, as shown in the cross-sectional focused ion beam (FIB) images, these changes were limited to the outermost surface (≥10 nm), and the plasma did not cause subsurface damage in any experimental group. In contrast to the vacuum plasma, the grain boundaries were clearly visible in the atmospheric pressure plasma, and some small grains at the grain boundaries were observed at longer exposure times.

### 3-4. Confirmation of Phase Transformation

X-ray diffraction (XRD) data were analyzed by the Rietveld method. The Rietveld refinement results indicated that all experimental groups consisted of four different crystal phases: tetragonal′, tetragonal, cubic and monoclinic phases. In order to confirm the phase transformation affected by the plasma treatment conditions, XRD patterns of all experimental groups in the 20 range of 27° to 31° are shown in A of FIG. 5. In the A1 and V1 experimental groups, asymmetric broadening and intensity decrease of the square peak (011) t are observed, which suggests the overlapping of two peaks (the tetragonal peak (011) t at 2=30.17° and the cubic peak (011)c at 2θ=30.07° (JCPDS card 27-0997). The quantitative phase composition as a function of exposure time inferred from the Rietveld analysis is shown in B and C of FIG. 5. The control group was mainly composed of tetragonal and cubic phases. In Rietveld improvement, the cubic phase fraction increased significantly after 1 minute of plasma exposure and further decreased to the control level as the exposure time increased. D of FIG. 5 shows changes in the unit cell parameter (Å) of the cubic phase according to the plasma exposure time, and the highest increase in the unit cell volume was observed in the A1 group.

A of FIG. 5 shows the X-ray diffraction patterns of all experimental groups, and B and C of FIG. 5 show the Rietveld quantitative analysis results as a function of plasma exposure time for the vacuum plasma treatment group (B) and the atmospheric pressure plasma treatment group (C). D of FIG. D shows the unit cell parameter (Å) of the cubic phase as a function of plasma exposure time.

### 3-5. Confirmation of Changes in Zeta Potential

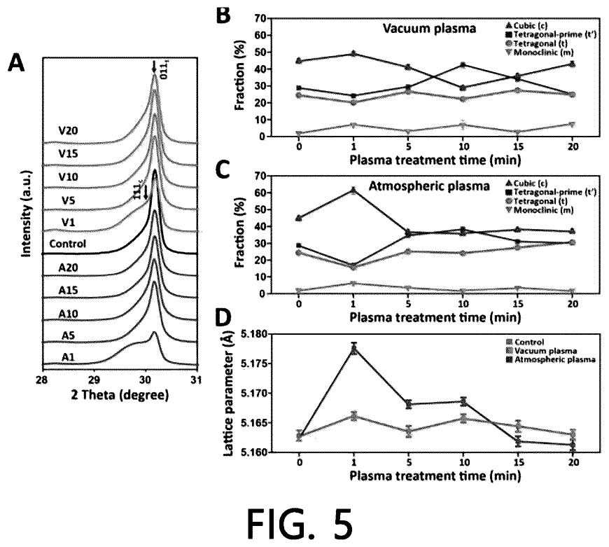

Two-way ANOVA showed that there was a statistically significant interaction between chamber pressure and plasma treatment time on surface zeta potential (p<0.05). Changes in the zeta potential according to treatment time using vacuum and atmospheric pressure plasma systems is shown in FIG. 6. The untreated sample (control) showed a negative zeta potential (−28.44 mV), and plasma treatment increased the zeta potential of 3Y-TZP. In the vacuum plasma treatment group (V5, V10, V15 and V20), the samples showed positive zeta potential values, which is presumed to be due to an increase in the basic hydroxyl (OH)b group on the surface. In the case of atmospheric pressure plasma, the zeta potential value weakened from a negative value with the treatment time. The zeta potential suddenly increased after 1 minute of treatment and increased slightly as the exposure time increased.

FIG. 6 shows changes in zeta potential according to the plasma treatment time using atmospheric pressure plasma and vacuum plasma. The zeta potentials of the control, atmospheric pressure plasma treatment and V1 were negative, whereas the zeta potentials of the vacuum plasma group except V1 were positive.

As a result, the plasma treatment under vacuum conditions changed all of the surface energy, chemical composition and zeta potential of 3Y-TZP. Therefore, the results according to the present invention confirm that the plasma treatment of 3Y-TZP is an effective method for biomedical and clinical applications.

Atmospheric pressure plasma treatment increased the electron donating (γ−) capacity of zirconia due to increased oxygen adsorption, whereas vacuum plasma treatment decreased the γ− parameter with increasing treatment time. Higher concentrations of reactive oxygen species were found in the atmospheric pressure plasma group compared to the vacuum plasma treatment group, which is related to the absorption of oxygen in the air. The highest percentage of OH(b) groups was obtained after 5 minutes of exposure to atmospheric pressure plasma. With longer exposure times, vacuum plasma induced physical or electrical damage, but this was limited to the outermost layers (>10 nm). Both plasma treatment groups increased the zeta potential of 3Y-TZP, which showed a positive value in vacuum. At atmospheric pressure, the zeta potential increased rapidly after 1 minute of exposure, and a slight increase was observed with longer exposure times. In the vacuum plasma system, much higher conversion and/or energy efficiencies could be achieved with shorter exposure times (<1 minute). However, longer exposure times could induce undesirable surface melting or decomposition. The enhanced surface functionalization of 3Y-TZP can be obtained after exposure to air for 1 to 5 minutes, by considering the amount of OH(b), polar components and nitrogen fixation involved in the plasma process.

The above description of the present invention is for illustrative purposes, and those skilled in the art to which the present invention pertains will understand that it can be easily modified into other specific forms without changing the technical idea or essential features of the present invention. Therefore, it should be understood that the exemplary embodiments described above are exemplary in all respects and not restrictive.

1. A 3Y-TZP zirconia material whose surface is modified by plasma treatment.

2. The 3Y-TZP zirconia material of claim 1, wherein the 3Y-TZP zirconia material is for dental use.

3. The 3Y-TZP zirconia material of claim 1, wherein the plasma is radiated under vacuum conditions.

4. The 3Y-TZP zirconia material of claim 1, wherein the plasma comprises N₂ or N₂/Ar as a carrier gas.

5. The 3Y-TZP zirconia material of claim 4, wherein the mixed gas of N₂/Ar comprises N₂ and Ar at a molar ratio of 1 to 5:5 to 50.

6. The 3Y-TZP zirconia material of claim 1, wherein the 3Y-TZP zirconia material is treated with plasma for 0.1 seconds to 10 minutes.

7. An implant made of a 3Y-TZP zirconia material whose surface is modified by plasma treatment.

8. The implant of claim 7, wherein the plasma is radiated under vacuum conditions.

9. The implant of claim 7, wherein the plasma comprises N₂ or N₂/Ar as a carrier gas.

10. A method for modifying the surface of a 3Y-TZP zirconia material, comprising the steps of:
(a) filling a carrier gas into a plasma generating device;
(b) generating plasma in the plasma generating device; and
(c) irradiating the generated plasma onto zirconia.

11. The method of claim 10, wherein the 3Y-TZP zirconia material is for dental use.

12. The method of claim 10, wherein the carrier gas is N₂ or N₂/Ar.

13. The method of claim 10, wherein the plasma generated in step (b) is radiated under vacuum conditions.

14. The method of claim 10, wherein the plasma of step (c) is irradiated to the zirconia for 0.1 seconds to 10 minutes.

15. A method for manufacturing a dental implant whose surface is modified, comprising the steps of:
(a) preparing an implant composed of a 3Y-TZP zirconia material;
(b) filling a plasma generating device with a carrier gas;
(c) generating plasma in the plasma generating device; and
(d) irradiating the generated plasma to the implant of step (a).

16. The method of claim 15, wherein the carrier gas is N₂ or N₂/Ar.

17. The method of claim 15, wherein the plasma of step (c) is generated by supplying a voltage of 0.1 kV to 40 kV and a frequency of 10 to 30 kHz to the plasma generating device.

18. The method of claim 15, wherein the plasma generated in step (c) is radiated under vacuum conditions.

19. The method of claim 15, wherein the plasma of step (d) is irradiated to the implant for 0.1 seconds to 10 minutes.
